(19) United States
(12) Patent Application Publication
(10) Pub. No.: US 2026/0090751 A1
(43) Pub. Date: Apr. 2, 2026

(54) METHOD AND SYSTEM FOR CARING A PSYCHOLOGICAL STATE USING BALLISTOCARDIOGRAM MEASURED THROUGH PIEZOELECTRIC ELEMENTS

(71) Applicant: DOLBOMDREAM CO. LTD., Daejeon (KR)

(72) Inventors: Jihun Kim, Goyang-si (KR); Seokhyun Kim, Daejeon (KR)

(73) Assignee: DOLBOMDREAM CO. LTD., Daejeon (KR)

(21) Appl. No.: 19/412,924

(22) Filed: Dec. 9, 2025

Related U.S. Application Data
(63) Continuation-in-part of application No. PCT/KR2024/005760, filed on Apr. 29, 2024.

(30) Foreign Application Priority Data
Jun. 16, 2023 (KR) 10-2023-0077741
Jun. 16, 2023 (KR) 10-2023-0141200

(51) Int. Cl. A61B 5/16; A61B 5/00; A61B 5/08; A61B 5/11

(52) U.S. Cl. CPC A61B 5/165; A61B 5/0816; A61B 5/1102; A61B 5/6804; A61B 5/7257; A61B 5/726; A61B 5/7264; A61B 2560/0204

(57) ABSTRACT

A wearable device includes: an air tube; an air inlet provided at one end of the air tube to inject or discharge air from the air tube; a driving unit configured to inject or discharge air into or from the air tube through the air inlet; a silicone tube disposed inside a housing of the driving unit and connected to the air inlet; a sensor module positioned inside the silicone tube and including a piezoelectric element configured to sense pressure; a first communication unit; a first artificial intelligence (AI) model unit; and a first processor disposed inside the silicone tube and operably connected to the driving unit, the sensor module, and the first AI model unit.

### CROSS REFERENCE TO PRIOR APPLICATIONS

This application is a continuation application of National Stage Application of PCT International Patent Application No. PCT/KR2024/005760 filed on Apr. 29, 2024, under 35 U.S.C. § 371, which claims priority to Korean Patent Application Nos. 10-2023-0077741 filed on Jun. 16, 2023, and 10-2023-0141200 filed on Jun. 16, 2023 which are all hereby incorporated by reference in their entirety.

### BACKGROUND

The present disclosure relates to a method and system for caring a psychological state of a user by applying deep-pressure stimulation to the user through an air-inflatable garment. More particularly, the disclosure relates to a technique for injecting air into the inflatable garment based on ballistocardiogram (BCG) information measured through a piezoelectric element provided in the garment.

With the increasing importance of managing and stabilizing a person's psychological state or stress level, there has been a growing need for devices and methods capable of alleviating anxiety or reducing stress.

In relation thereto, various methods and technologies have been proposed in which a wearable device including a biometric sensor acquires biometric information of a user, and the user's psychological state is estimated or determined based on the acquired biometric information.

### SUMMARY

In order to determine or estimate the psychological state or stress level of a user, biometric information of the user is required. Moreover, for accurate determination, the biometric information serving as the basis for evaluating psychological or stress conditions must include minimal noise. Thus, there is a need for a method capable of acquiring the user's biometric information in real time while minimizing noise.

A wearable device equipped with a biometric sensor and an air tube for caring a user's psychological condition has also been proposed as a solution for collecting biometric information and providing psychological care without restricting the user to a specific space. Specifically, while the user wears the device in an unrestricted environment, biometric information is collected in real time, and air is injected into the air tube based on the collected information to provide psychological care.

However, conventional systems require that the biometric information collected through the wearable device be transmitted to an external server, the external server determines whether air should be injected into the air tube, and the wearable device then receives control data including the server's determination.

Accordingly, the control of the wearable device depends on receiving control data from the external server, making a communication link between the device and the server essential.

In practice, the communication condition between the wearable device and the external server may become unstable depending on the user's physical environment. In situations where communication cannot be established, the wearable device becomes unable to operate properly.

Therefore, there is a need for a solution that enables the wearable device to operate independently of external server communication by utilizing a control system embedded within the wearable device itself.

According to one embodiment, a system for caring a psychological state of a user based on the user's biometric information may include a wearable device and an external server. The wearable device may include an air tube, an air inlet formed at one end of the air tube to allow air to be injected into or discharged from the air tube, a driving unit configured to inject or discharge air into or from the air tube through the air inlet, a silicon tube disposed within a housing of the driving unit and extending from the driving unit to the air inlet, a sensor module located inside the silicon tube and including a piezoelectric element configured to detect pressure, a first communication unit, a first artificial intelligence (AI) model unit, and a first processor located within the silicon tube and operably connected to the driving unit, the sensor module, and the first AI model unit.

The external server may include a second communication unit, a second AI model unit, and a second processor operably connected to the second communication unit and the second AI model unit.

The first processor acquires air pressure data representing air pressure inside the air tube through the piezoelectric element of the sensor module, determines whether a network connection is established such that data communication between the wearable device and the external server is available, and, when the network connection is determined to be established, transmits the air pressure data to the external server through the first communication unit.

The second processor receives the air pressure data from the wearable device through the second communication unit, inputs the air pressure data into a first AI model and a second AI model included in the second AI model unit, updates the first AI model by training the model to extract filtered data from the air pressure data, updates the second AI model by training the model to determine the user's state, and, when the network connection is determined to be established, transmits the updated first AI model and the updated second AI model to the wearable device through the second communication unit.

The first processor receives the updated first AI model and the updated second AI model from the external server through the first communication unit, and determines the user's state based on values obtained by processing the air pressure data through the updated first AI model and the updated second AI model.

According to an embodiment, biometric information for determining a user's psychological state or stress level may be acquired based on air pressure data measured by a piezoelectric element provided inside a silicon tube within the driving unit of the wearable device. As a result, the biometric information may include significantly less noise compared to conventional biometric information acquired through typical biometric sensors.

Therefore, by using a wearable device equipped with a sensor module including a piezoelectric element embedded within a silicon tube, the system may obtain biometric information having substantially reduced noise, determine or estimate a user's psychological state and stress level with improved accuracy, and effectively reduce the user's anxiety or stress by controlling the wearable device to apply deep-pressure stimulation to the user.

Additional direct and indirect advantages may also be provided through the present disclosure.

### BRIEF DESCRIPTIONS OF THE DRAWINGS

FIG. 1 illustrates a system for caring a psychological state of a user using a wearable device according to an embodiment.

FIG. 2 illustrates a wearable device including a sensor module configured to measure ballistocardiogram (BCG) according to an embodiment.

FIG. 3 illustrates an internal configuration of a sensor module configured to measure BCG according to an embodiment.

FIG. 4 illustrates a block diagram of a sensor module configured to measure BCG according to an embodiment.

FIG. 5 illustrates a conceptual diagram showing a technique for acquiring biometric information of a user using a sensor module provided in the wearable device according to an embodiment.

FIG. 6 illustrates a conceptual diagram showing how the sensor module acquires and processes raw data according to an embodiment.

FIG. 7A illustrates a graph for explaining a Fourier transform performed during preprocessing of raw data acquired by the sensor module according to an embodiment.

FIG. 7B illustrates a graph for explaining a wavelet transform performed during preprocessing of raw data acquired by the sensor module according to an embodiment.

FIG. 8A illustrates a flow of template matching performed by a first artificial intelligence model according to an embodiment.

FIG. 8B illustrates a graph showing an example of template matching performed by the first artificial intelligence model according to an embodiment.

FIG. 9 illustrates an overall flow of operations of a system including a wearable device and a server according to an embodiment.

FIG. 10 illustrates a flowchart of a method for caring a psychological state of a user using biometric information according to an embodiment.

FIG. 11 illustrates a flowchart of operations between the wearable device and the server for caring a psychological state of a user using biometric information according to an embodiment.

FIG. 12 illustrates a flowchart of operations between the wearable device and the server for updating an artificial intelligence model according to an embodiment.

In the drawings, identical or similar reference numerals may be used to denote identical or similar elements.

### DETAILED DESCRIPTION OF THE INVENTION

The various embodiments of the present disclosure will now be described in detail with reference to the accompanying drawings. However, the present disclosure is not limited to the specific embodiments described herein, and it should be understood that the embodiments encompass various modifications, equivalents, and/or alternatives without departing from the spirit and scope of the invention.

The embodiments described below are provided so that those skilled in the art may readily understand and implement the invention. The present disclosure may be embodied in many different forms and should not be construed as limited to the embodiments set forth herein. In the drawings, components irrelevant to the description of the invention may be omitted for clarity, and identical reference numerals may be used throughout the specification to refer to identical or similar elements.

Throughout the specification, when an element is described as “including” or “comprising” another element, this indicates that the element may include additional elements unless explicitly stated otherwise. The terms “unit,” “device,” and “module,” as used herein, refer to functional blocks configured to perform at least one operation or function, and may be implemented in hardware, software, or a combination of hardware and software.

Throughout the specification, when an element is described as being “connected” to another element, this includes not only a direct connection but also an indirect electrical connection through one or more intervening elements, unless otherwise specified. Furthermore, when an element is described as “including” another component, this does not exclude the presence of additional components or features, and the presence or addition of one or more other features, numbers, steps, operations, elements, or components is not precluded.

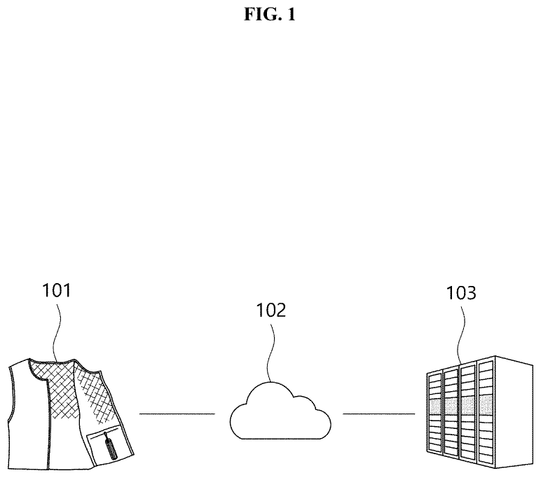

FIG. 1 illustrates a system for caring a psychological state of a user using a wearable device 101 according to an embodiment. Referring to FIG. 1, the system may include the wearable device 101, a network 102, and a server 103. However, the system is not limited to the components illustrated in FIG. 1, and certain components may be omitted or additional components may be included. For example, the system may further include a user terminal, such as a smartphone.

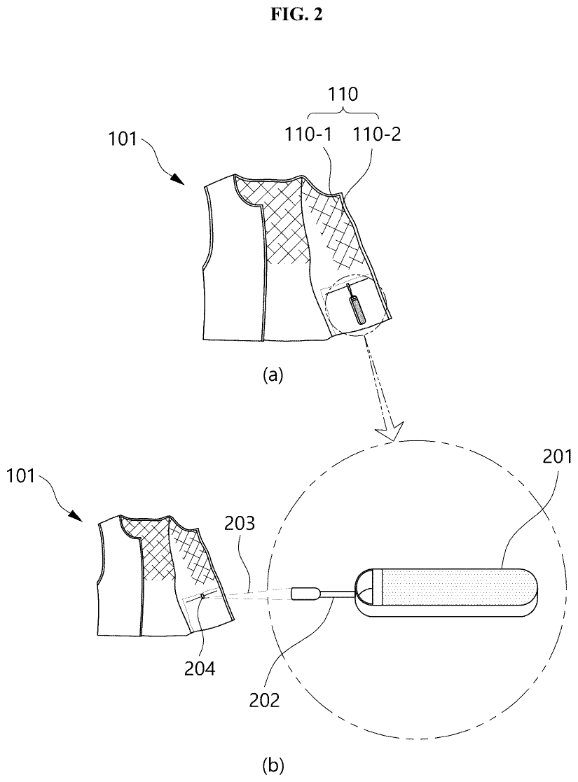

According to one embodiment, the wearable device 101 may be an air-inflatable garment including an air tube (e.g., air tube 110 of FIG. 2), an air inlet (e.g., air inlet 204 of FIG. 2), and a driving unit configured to provide deep-touch pressure (DTP) to a user wearing the device. For example, the wearable device 101 may be designed to deliver deep-pressure stimulation to alleviate psychological anxiety or reduce stress for a user experiencing heightened anxiety or tension. The term “deep-touch pressure” refers to a type of pressure applied to the user's body that stimulates the parasympathetic nervous system, providing a sensation similar to being hugged, thereby reducing anxiety and promoting psychological stability. The user may include an individual requiring emotional stabilization or stress relief, such as a child or adult with developmental disabilities. However, the user is not limited to such examples and may include infants, children, adolescents, disabled individuals, or elderly individuals.

According to an embodiment, the driving unit may function as an air pump that injects air into the air tube 110 through the air inlet 204 or extracts air therefrom. For example, when the user wearing the wearable device 101 is determined to be experiencing anxiety or stress, the driving unit may inject air into the air tube through the air inlet. When it is determined that the user's anxiety or stress has subsided, the driving unit may extract air from the air tube. Whether the user is experiencing anxiety or stress may be determined based on biometric information acquired through a sensor module (e.g., sensor module 201) included in the wearable device 101. For example, when a value included in the biometric information exceeds a preset value and/or deviates from a predefined range, the wearable device 101 may determine that the user is anxious or stressed.

According to one embodiment, the wearable device 101 may include a communication module enabling data communication with an external electronic device. For example, the wearable device 101 may communicate with the server 103 through the network 102. The network 102 may include a wireless communication network, such as a mobile hotspot or Wi-Fi.

According to an embodiment, the wearable device 101 may transmit biometric information—such as ballistocardiogram (BCG) information, heart rate variability (HRV) information, or respiration information—acquired through the sensor module (e.g., FIG. 2, element 201) to the server 103 through the network 102. The wearable device 101 may also receive an updated artificial intelligence (AI) model from the server 103.

According to an embodiment, the server 103 may communicate with the wearable device 101 and may operate an AI model configured to compute control values for the driving unit of the wearable device 101. For example, the server 103 may train the AI model using training data and obtain output data by inputting data into the model.

According to another embodiment, when data communication between the server 103 and the wearable device 101 is established, the server 103 may receive biometric information from the wearable device 101. When the communication link is established, the server 103 may transmit an updated AI model to the wearable device 101.

Although not illustrated in FIG. 1, the wearable device 101 may also communicate with a user terminal, such as a smartphone, through the network 102. For example, the wearable device 101 may transmit biometric information acquired through the sensor module to the user terminal. The user terminal may transmit command data to control the wearable device 101 through the network 102.

FIG. 2 illustrates a wearable device 101 including a sensor module 201 configured to measure ballistocardiogram (BCG) according to an embodiment. Referring to FIG. 2, the wearable device 101 may include the sensor module 201 having a piezoelectric element configured to measure BCG, an air tube 110 including a first adhesive portion 110-1 of a first type and a second adhesive portion 110-2 of a second type, an air inlet 204, and a driving unit (not shown).

According to one embodiment, although not illustrated in FIG. 2, the wearable device 101 is not limited to a sensor module including a piezoelectric element for measuring BCG and may further include one or more biometric sensors capable of measuring additional biometric information. For example, the biometric sensors may include an electrodermal activity (EDA) sensor, a photoplethysmography (PPG) sensor, a blood volume pulse (BVP) sensor, or a thermal (or body-temperature) sensor.

In the embodiments described below, the wearable device 101 is assumed to be a vest-type garment equipped with the air tube 110. However, the configuration of the wearable device 101 is not limited to a vest, and may include any garment capable of providing deep-touch pressure to the user. In one embodiment, the wearable device 101 may include a front portion that contacts the user's chest and a back portion that contacts the user's back when worn.

According to one embodiment, the wearable device 101 may include the first adhesive portion 110-1 and the second adhesive portion 110-2 such that air injected through the driving unit applies pressure uniformly across the user's chest and back. The first adhesive portion 110-1 may be formed as a point-shaped or circular adhesive region, whereas the second adhesive portion 110-2 may be formed as a line-shaped adhesive region of a predetermined length. By alternately arranging the first adhesive portion 110-1 and the second adhesive portion 110-2, the air tube 110 may form an air flow path for distributing injected air. In one embodiment, the alternating arrangement of the adhesive portions forms a continuous adhesive line, and a plurality of such adhesive lines may be arranged laterally across the front and back portions of the wearable device 101. The second adhesive portion 110-2 may be straight or may have a curved shape defined by a predetermined curvature.

According to one embodiment, the sensor module 201 may include a piezoelectric element disposed inside a housing and may include a silicon tube 202 configured to seal the piezoelectric element. For example, the silicon tube 202 may accommodate the piezoelectric element or a printed circuit board (PCB, or BCB signal board) on which the piezoelectric element is mounted such that the entire PCB is sealed within the silicon tube.

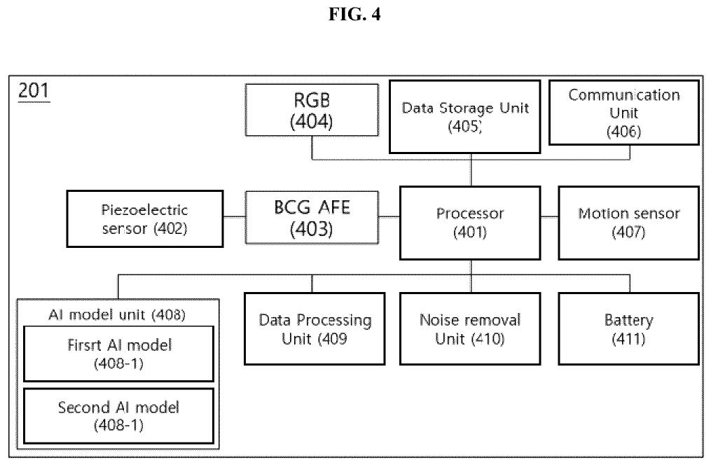

According to an embodiment, the air tube 110 of the wearable device 101 may be configured as a sealed structure except for the air inlet 204 through which air is injected or discharged. An end of the silicon tube 202 extending from the sensor module 201 may be coupled (203) to the air inlet 204 of the wearable device 101. The silicon tube 202 extending from the sensor module 201 may be sealed except for the end portion connected to the air inlet 204. Thus, when the end of the silicon tube 202 is connected to the air inlet 204, pressure of the air sealed inside the air tube 110 and the silicon tube 202 may be applied to the piezoelectric element. The piezoelectric element may detect air-pressure variations of the wearable device 101 caused by the user's heartbeats and/or respiration. The processor of the wearable device 101 (e.g., processor 401 of FIG. 4) may acquire BCG data based on the air-pressure variation data detected by the piezoelectric element.

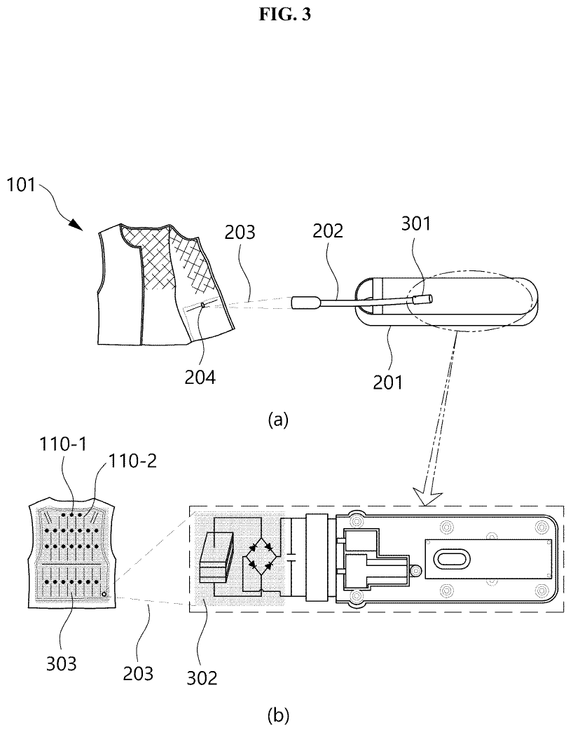

FIG. 3 illustrates an internal configuration of a sensor module 201 for measuring ballistocardiogram (BCG) according to an embodiment. Referring to FIG. 3, the sensor module 201 may include a piezoelectric element, and a silicon tube 202 may be disposed to seal 302 (or hermetically enclose) the piezoelectric element. The wearable device 101 may include an air tube 110 forming a sealed (or hermetically enclosed) space 303 in which air is injected across the entire front and back portions of the wearable device 101. The air tube 110 may include a first-type air adhesive portion 110-1 and a second-type air adhesive portion 110-2.

According to one embodiment, when the silicon tube 202 of the sensor module 201 is connected to the air inlet 204 of the wearable device 101, the piezoelectric element of the sensor module 201 detects variations in air pressure within the air tube 110 caused by the user's heartbeats and/or respiration when the wearable device 101 is worn.

According to another embodiment, a processor of the sensor module 201 (e.g., processor 401 of FIG. 4) amplifies a minute output voltage generated by the piezoelectric element by using an amplifier such as a BCG analog front end (AFE) 403, and filters the amplified signal to generate an analog voltage suitable for analog-to-digital conversion (ADC) performed by the processor.

FIG. 4 illustrates a block diagram of a sensor module 201 for measuring ballistocardiogram (BCG) signals according to an embodiment.

Referring to FIG. 4, the sensor module 201 may include a processor 401, a piezoelectric element 402, a BCG analog front end (AFE) 403, an RGB indicator 404, a data storage unit 405, a communication unit 406, a motion sensor 407, an artificial intelligence (AI) model unit 408, a data processing unit 409, a noise removal unit 410, and a battery 411. However, the sensor module 201 is not limited to the components illustrated in FIG. 4, and certain components may be omitted or additional components may be further included. For example, the RGB indicator 404 may be omitted. As another example, the sensor module 201 may further include a pressure sensor capable of detecting an air pressure inside the air tube 110 of the wearable device 101.

The AI model unit 408 may include a first AI model 408-1 and a second AI model 408-2. The first AI model 408-1 may process (or pre-process) air-pressure data or biometric signals acquired through the piezoelectric element 402. The second AI model 408-2 may classify a user's symptoms based on processed data (e.g., HRV data) derived from the data (e.g., BCG data) output by the first AI model 408-1.

In one embodiment, although not explicitly shown in FIG. 4, the sensor module 201 may be operably and/or electrically connected to a driving unit configured to inject or extract air into or from the air tube 110 of the wearable device 101. For example, the sensor module 201 may be disposed inside a housing of the driving unit.

In another embodiment, although not shown in FIG. 4, the battery 411 may also be operably and/or electrically connected to the driving unit. For example, the battery 411 may supply power to the driving unit. The battery 411 may be separate from the sensor module 201 and may supply power to both the sensor module 201 and the driving unit.

In another embodiment, although not illustrated in FIG. 4, the sensor module 201 may be positioned inside a silicone tube, and the sensor module 201 located inside the silicone tube may be disposed within a housing of the driving unit.

In one embodiment, the processor 401 may be a dedicated processor for controlling a specific system, such as a microcontroller unit (MCU). The processor 401 may be operably and/or electrically connected to the piezoelectric element 402, the BCG AFE 403, the RGB indicator 404, the data storage unit 405, the communication unit 406, the motion sensor 407, the AI model unit 408, the data processing unit 409, the noise removal unit 410, and the battery 411. The processor 401 may analyze and process various types of data obtained from these components and control the corresponding components based on the analysis and processing results.

In one embodiment, the processor 401 acquires air-pressure data inside the air tube 110 of the wearable device 101 through the piezoelectric element 402.

In one embodiment, the BCG AFE 403 is a circuit configured to amplify and filter a minute output voltage from the piezoelectric element 402 and to generate an analog voltage that enables the processor 401 to perform analog-to-digital conversion (ADC).

In one embodiment, the RGB 404 may be a three-color LED that indicates an operating state of the processor 401.

In one embodiment, the data storage unit 405 stores the air-pressure data acquired through the piezoelectric element 402 and stores output data generated by the AI model unit 408.

In one embodiment, the communication unit 406 supports data communication with an external device, such as the server 103. For example, the wearable device 101 may transmit biometric signals to the server 103 through the communication unit 406.

In one embodiment, the motion sensor 407 acquires motion data of a user wearing the wearable device 101. For example, the motion sensor 407 may include a three-axis motion sensor, an accelerometer, a geomagnetic sensor, and/or a gyroscope.

In one embodiment, the motion sensor 407 performs I2C communication with the processor 401. The motion sensor 407 provides the user's motion data acquired by the motion sensor 407 to the processor 401 through I2C communication.

In one embodiment, the AI model unit 408 includes a first AI model 408-1 and a second AI model 408-2. The first AI model 408-1 processes (or pre-processes) air-pressure data or biometric signals (e.g., BCG signals) acquired through the piezoelectric element 402. That is, the first AI model 408-1 is an AI model that performs a raw-data filter using a template-matching technique to extract filtered data (filtered signals) from raw data that has been processed (e.g., Fourier-transformed and wavelet-transformed). The first AI model 408-1 may be referred to as a similarity-signal matching model based on the template-matching technique. The raw data may be the air-pressure data acquired through the piezoelectric element 402 or the air-pressure data pre-processed by the data processing unit 409. The pre-processing may include Fourier transformation and wavelet transformation.

In one embodiment, the first AI model 408-1 is trained to output filtered data when receiving raw data as input by using a normal BCG data set (or normal biometric data set). The normal BCG data set or the normal biometric data set may serve as template data. The first AI model 408-1 may be trained such that the filtered data is output differently depending on individual users. The normal biometric data set may include at least one of a normal heart-rate data set, a normal heart-rate variability data set, a normal respiration-rate data set, a normal electrodermal activity data set, a normal photoplethysmography (PPG) data set, a normal blood-volume pulse data set, or a normal temperature data set.

In one embodiment, the first AI model 408-1 is an artificial intelligence model that receives, as input data, at least one of the air-pressure data acquired through the piezoelectric element 402 and pre-processed data of the air-pressure data generated by the data processing unit 409, and outputs a normal BCG data set (or a normal biometric data set).

In one embodiment, the second AI model 408-2 is an artificial intelligence model configured to classify a user's symptoms based on processed data (e.g., heart-rate variability (HRV) data) generated from the data output by the first AI model 408-1 (e.g., BCG data).

In one embodiment, the second AI model 408-2 is trained to output a type of symptom when receiving, as input, processed data (e.g., HRV data) generated from the BCG data output by the first AI model 408-1 by using HRV data corresponding to respective symptoms.

In one embodiment, the symptoms include stress-related symptoms, respiration-related symptoms, pain-related symptoms, depression-related symptoms, or cardiac-disease-related symptoms.

In one embodiment, stress-related symptoms may reduce HRV. For example, anxiety, tension, irritability, or physical stress may cause irregularity in heart-beat intervals and a decrease in HRV. The second AI model 408-2 may be trained using HRV data having reduced variability and irregular heart-beat intervals corresponding to stress-related symptoms.

In one embodiment, respiration-related symptoms such as altered breathing patterns, airway obstruction, asthma, or dyspnea may reduce HRV. The second AI model 408-2 may be trained using HRV data having reduced variability corresponding to respiration-related symptoms.

In one embodiment, pain-related symptoms such as chronic pain, neuralgia, or myalgia may cause irregular heart-beat intervals and a decrease in HRV. The second AI model 408-2 may be trained using HRV data showing reduced variability and irregularity corresponding to pain-related symptoms.

In one embodiment, depression-related symptoms such as depressed mood, lethargy, fatigue, or low affect may be associated with reduced HRV. The second AI model 408-2 may be trained using HRV data reduced in variability corresponding to depression-related symptoms.

In one embodiment, cardiac-disease-related symptoms such as heart failure, arrhythmia, or myocardial infarction may reduce the variability of HRV. The second AI model 408-2 may be trained using HRV data having reduced variability corresponding to cardiac-disease-related symptoms.

In one embodiment, the data processing unit 409 is a circuit configured to process sensing data (e.g., BCG raw data) acquired through a sensor such as the piezoelectric element 402 of the sensor module 201, into input data for the AI model (e.g., the first AI model 408-1). For example, the data processing unit 409 performs a pre-processing operation to convert air-pressure data representing the air pressure inside the air tube 110 of the wearable device 101, acquired through the piezoelectric element 402, into input data for the first AI model 408-1 of the AI model unit 408. The pre-processing may include Fourier transformation and wavelet transformation. The data processing unit 409 may apply a first Fourier transform to the air-pressure data and may subsequently apply a wavelet transform to the Fourier-transformed data.

In one embodiment, the noise-removal unit 410 removes noise included in the sensing data detected through the piezoelectric element 402 based on the motion data acquired from the motion sensor 407 (e.g., a three-axis motion sensor, accelerometer, geomagnetic sensor, and/or gyroscope). For example, the noise-removal unit 410 removes amplified signals by superimposing x-, y-, and z-axis signals measured by the accelerometer onto the sensing data (e.g., air-pressure data) measured through the piezoelectric element 402, thereby removing noise included in the sensing data.

In one embodiment, the battery 411 supplies power to operate the sensor module 201. The battery 411 may support wired and/or wireless charging. When the battery 411 is in a charging state, the communication unit 406 may be activated. For example, the processor 401 activates the communication unit 406 in response to identifying that the battery 411 is in the charging state, and thereby performs data communication with an external device such as the server 103. That is, when the battery 411 is in the charging state, the processor 401 transmits sensing data to the server 103 through the communication unit 406. The sensing data may include sensing data detected through the piezoelectric element 402 (e.g., air-pressure data) or processed data generated by the data processing unit 409 (e.g., data subjected to pre-processing including Fourier and wavelet transformations and data output by the first AI model 408-1).

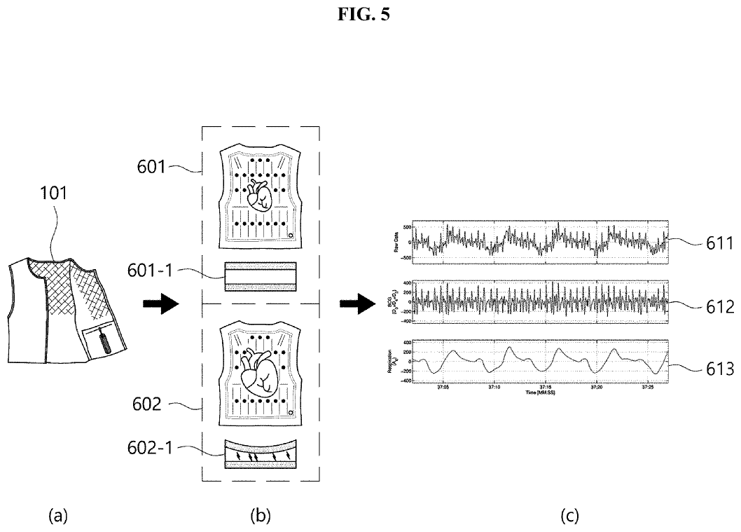

FIG. 5 illustrates a conceptual diagram of a technique for acquiring a user's biometric information using the sensor module 201 provided in the wearable device 101 according to an embodiment.

Referring to FIG. 5(a), a wearable device 101 equipped with a sensor (e.g., the sensor module 201) for acquiring the user's biometric information in real time when the device is worn by the user is shown. FIG. 5(b) illustrates contraction and expansion of the user's blood vessels depending on the user's biometric state (e.g., cardiac activity or respiratory state) when the wearable device 101 is worn. FIG. 5(c) illustrates a graph representing biometric information of the user wearing the wearable device 101, acquired in real time through the sensor of the wearable device 101.

In one embodiment, when the user wearing the wearable device 101 exhales or when the heart contracts (501), the user's blood vessels may remain in a normal state or may expand. When the user inhales or when the heart expands (502), the user's blood vessels may contract relative to the normal state.

In one embodiment, the wearable device 101 acquires the user's biometric information through the sensor module 201. The wearable device 101 acquires raw data 511 representing the user's biometric information through the sensor module 201. The wearable device 101 processes the raw data through the data processing unit 409 to generate ballistocardiogram (BCG) data 512 and respiration data 513. For example, the wearable device 101 may apply a Fourier transform and a wavelet transform to the raw data through the data processing unit 409. The wearable device 101 then inputs the transformed data, to which the Fourier transform and the wavelet transform have been applied, into the first AI model 408-1 to output filtered data. The filtered data may include BCG data 612 or respiration data 613.

In one embodiment, the Fourier transform refers to a process of separating frequencies corresponding to the cardiac rhythm and the respiratory rhythm from the raw data measured through the piezoelectric element 402.

In one embodiment, the wavelet transform may refer to a process of generating various frequency bands ranging from low frequencies to high frequencies by stretching or compressing the signal length along the time axis, and a process of transforming the raw data by varying the time scale of a wavelet function modeled according to predetermined rules and calculating correlation coefficients with the raw data.

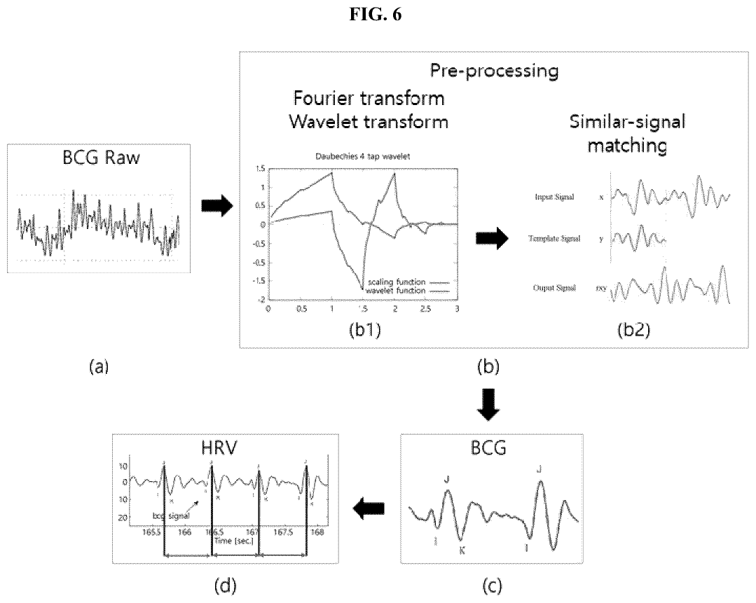

FIG. 6 illustrates a conceptual diagram of a technique in which the sensor module 201 acquires and processes raw data according to an embodiment.

Referring to FIG. 6, the wearable device 101 acquires BCG raw data through the sensor module 201 provided in the wearable device 101 (FIG. 6(a)), performs pre-processing on the BCG raw data (FIG. 6(b)), and then obtains data (e.g., heart rate variability (HRV) data) that serves as the basis for determining an abnormal state of the user through a series of data-processing operations (FIGS. 6(c) and 6(d)).

Referring to FIG. 6(a), the wearable device 101 acquires raw data through a piezoelectric element 402 inside the sensor module 201. The raw data may represent pressure data, and more specifically, may represent air-pressure data indicating an air pressure inside a silicone tube that surrounds the piezoelectric element 402.

In one embodiment, the piezoelectric element 402 is understood as an element configured to measure pressure, and the piezoelectric element 402 positioned inside the silicone tube senses a pressure applied by air inside the silicone tube. The silicone tube is connectable to the air tube 110 of the wearable device 101, and when connected, the silicone tube and the interior of the air tube 110 form a single sealed space.

That is, when the user inhales, exhales, or experiences cardiac activity while wearing the wearable device 101, the user's body (e.g., chest or back) applies pressure or relieves pressure on the air tube 110, thereby causing air-pressure changes inside the air tube 110. The changes in air pressure inside the air tube 110 cause corresponding changes in the internal air pressure of the silicone tube forming the sealed space. The piezoelectric element 402 detects the changes in the internal air pressure of the silicone tube and thereby effectively detects the air-pressure changes occurring inside the air tube 110.

Referring to FIG. 6(b), the wearable device 101 may pre-process the raw data through the data processing unit 409. As shown in FIGS. 6(b1) and 6(b2), the pre-processing may include a Fourier transform, a wavelet transform, and a similar-signal matching operation.

In one embodiment, the Fourier transform refers to a process of separating a frequency corresponding to a biological rhythm (e.g., a cardiac rhythm or a respiratory rhythm) from the raw data (e.g., pressure data or air-pressure data).

In one embodiment, the wavelet transform refers to a process of generating various frequency bands ranging from low frequencies to high frequencies by stretching or compressing the length of the signal along a time axis, and a process of performing a transform by changing the time scale of a modeled wavelet function according to predetermined rules and computing a correlation coefficient between the wavelet function and the raw data.

Referring to FIG. 6(b2), the similar-signal matching operation may indicate a process in which the wearable device 101 processes data using the first artificial intelligence (AI) model 408-1. The wearable device 101 uses the first AI model 408-1 of the AI model unit 408 to convert the data that has undergone the Fourier transform and the wavelet transform (e.g., second transformed data) into filtered data (e.g., biological data).

Referring to FIG. 6(c), the wearable device 101 applies a template-matching technique to the raw data using the first AI model 408-1 and outputs filtered data. The filtered data may include at least one of ballistocardiogram (BCG) data and respiration data.

Referring to FIG. 6(d), the wearable device 101 extracts heart-rate variability (HRV) data from the BCG data through the data processing unit 409.

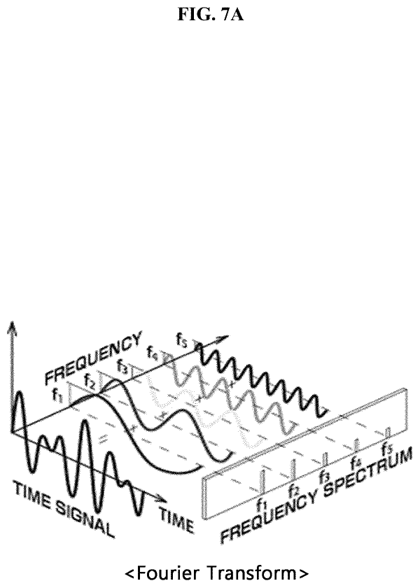

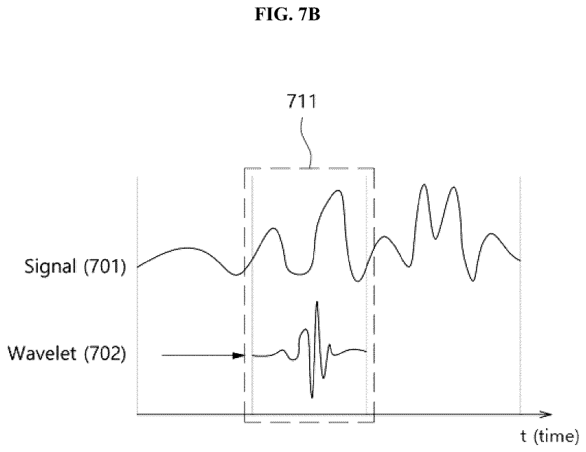

FIG. 7A illustrates a graph for explaining a Fourier transform performed during a pre-processing operation of raw data acquired by the sensor module 201 according to one embodiment, and FIG. 7B illustrates a graph for explaining a wavelet transform performed during a pre-processing operation of raw data acquired by the sensor module according to one embodiment.

Referring to FIG. 7A, the Fourier transform may refer to a process of separating a frequency corresponding to a biological rhythm (e.g., a cardiac rhythm or a respiratory rhythm) from raw data (e.g., pressure data or air-pressure data) acquired through the piezoelectric element 402. For example, the wearable device 101 may separate the frequency corresponding to the cardiac rhythm or respiratory rhythm from the raw data through the data processing unit 409 and generate first transformed data. The frequency corresponding to the cardiac rhythm or respiratory rhythm may correspond to one of a plurality of frequency spectra included in the raw data. The wearable device 101 may apply the Fourier transform to the raw data through the data processing unit 409 to generate the first transformed data.

Referring to FIG. 7B, the wavelet transform may refer to a process of generating various frequency bands ranging from low frequencies to high frequencies by stretching or compressing the length of a signal 701 along a time axis, and a process of performing a transform by changing a time scale of a modeled wavelet function according to predetermined rules and computing a correlation coefficient between the raw data and the wavelet function. For example, the wearable device 101 may apply the wavelet transform to the signal 701 (e.g., raw data) such that a frequency of the signal 701 over the entire time range is converted into a wavelet signal 702 corresponding to a specific frequency band 711.

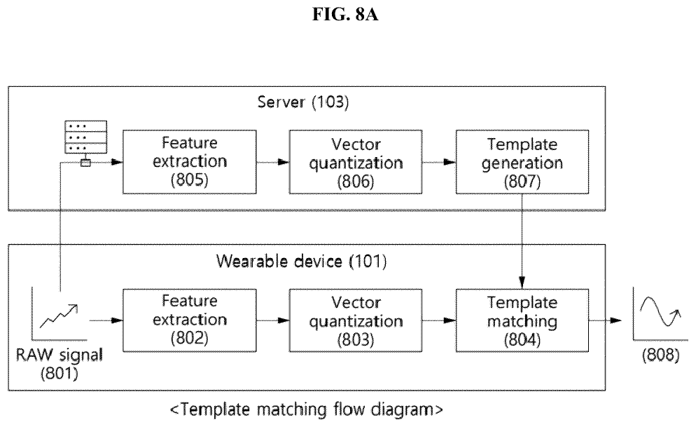

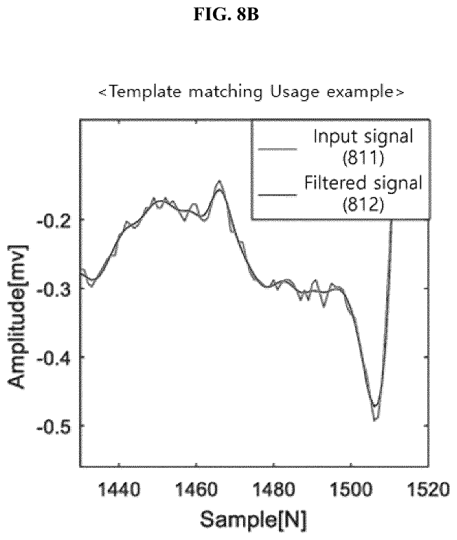

FIG. 8A illustrates a flowchart of template matching performed by the first artificial intelligence (AI) model 408-1 according to one embodiment, and FIG. 8B illustrates a graph showing an example of template matching performed by the first AI model 408-1 according to one embodiment.

Referring to FIG. 8A, the wearable device 101 may output data 808, to which the raw-data filter has been applied (e.g., filtered BCG data or filtered respiration data), by applying a template matching technique to raw data 801 using the first AI model 408-1.

In one embodiment, the wearable device 101 may acquire raw data 801 (or raw signals) through the sensor module 201 (or the piezoelectric element 402). The wearable device 101 may transmit the raw data 801 to the server 103.

In one embodiment, the server 103 may receive the raw data 801 from the wearable device 101. The server 103 may extract features 805 from the raw data 801. Based on the extracted features, the server 103 may perform vector quantization 806. Based on the vector quantization, the server 103 may generate a template 807. The template refers to template data used by the wearable device 101 to perform the template matching process of the first AI model 408-1, and the data input to the first AI model 408-1 may be filtered based on the template data. The template may vary according to a user's personal information (e.g., age, sex, height, weight, and disability level) and biometric information. The server 103 may transmit the generated template (or template data) to the wearable device 101.

In one embodiment, the wearable device 101 may extract features 802 from the raw data 801 and perform vector quantization 803. Using the template (or template data) received from the server 103, the wearable device 101 may apply the first AI model 408-1 to the vector-quantized data and output filtered data 808. The filtered data 808 may be data with reduced noise compared to the raw data 801. For example, referring to FIG. 8B, the wearable device 101 may output a filtered signal 812 by applying a template matching technique to an input signal 811, and the filtered signal 812 may have reduced noise relative to the input signal 811.

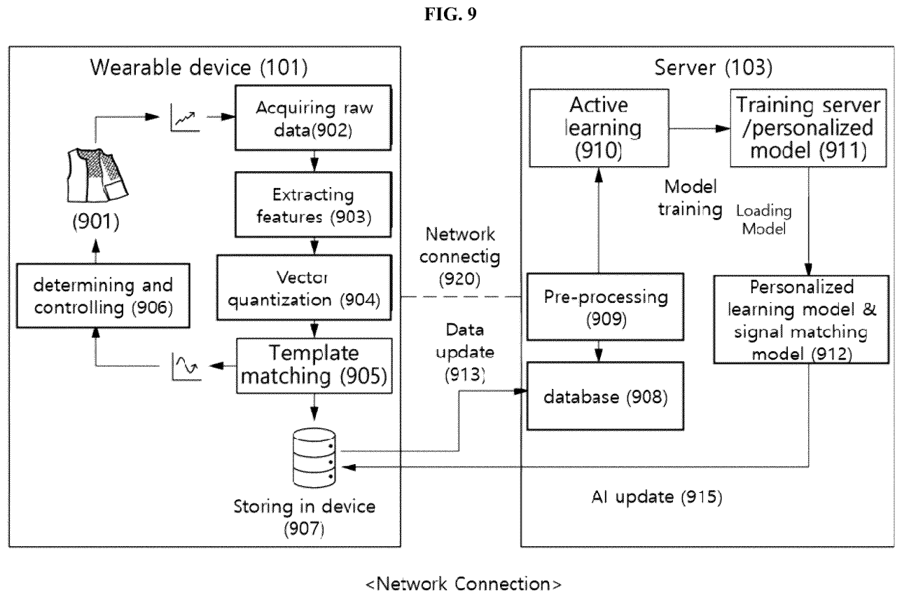

FIG. 9 illustrates a diagram for explaining an overall operation flow of a system including the wearable device 101 and the server 103 according to one embodiment.

Referring to FIG. 9, when a network connection 920 between the wearable device 101 and the server 103 is established, the wearable device 101 may transmit data acquired by the wearable device 101 (e.g., raw data, transformed data, filtered data, and biometric data) to the server 103. The wearable device 101 may also receive an artificial intelligence (AI) model updated by the server 103 and control the wearable device 101 using the updated model.

In step 901, the wearable device 101 may be worn by a user, and the silicone tube inside the actuator of the wearable device 101 may be connected to the air tube 110, such that the air tube 110 and the entire interior of the silicone tube form a single sealed space.

In step 902, in the state of step 901, the wearable device 101 may acquire raw data through the sensor module 201.

In step 903, the wearable device 101 may extract features from the raw data. The feature extraction process may correspond to a preprocessing operation for enabling the raw data to be processed by an AI model.

In step 904, the wearable device 101 may perform vector quantization on the raw data. The vector quantization process may also correspond to a preprocessing operation for enabling the raw data to be processed by an AI model.

In step 905, the wearable device 101 may perform template matching on the preprocessed data (e.g., second transformed data) based on the template (or template data) received from the server 103, and may output filtered data.

In step 907, the wearable device 101 may store the output data in the data storage unit 405. Although not illustrated in FIG. 9, the wearable device 101 may store, in the data storage unit 405, not only the filtered data but also at least one of the raw data, first transformed data, second transformed data, or filtered data.

In step 908, only when the wearable device 101 and the server 103 are network-connected 920, the wearable device 101 may transmit to the server 103 the data stored in the data storage unit 405 (e.g., raw data, first transformed data, second transformed data, or filtered data). The server 103 may store the received data in a database.

In step 909, the server 103 may perform preprocessing on the data to train an AI model. The preprocessing may be substantially identical to the operations performed in steps 903 and 904. For example, the server 103 may extract features from the data stored in the database and perform vector quantization.

In step 910, the server 103 may perform active learning on the AI model operating in the server 103 using the preprocessed data. The AI model may be substantially identical to the first AI model 408-1 and the second AI model 408-2 operating in the wearable device 101, and the learning method and inference method may also be substantially identical.

In one embodiment, active learning enables the AI model to autonomously select training data and expand the training dataset during the learning process, thereby reducing data labeling costs and enabling the construction of an effective AI model even with a relatively small amount of labeled data.

In step 911, the server 103 may train the AI model operating in the server 103 using active learning.

In step 912, the server 103 may load the trained AI model based on the data received from the wearable device 101.

In step 915, only when the server 103 and the wearable device 101 are network-connected 920, the server 103 may transmit the trained AI model to the wearable device 101. Accordingly, the wearable device 101 may operate based on the updated AI model received from the server 103. The operation of the wearable device 101 refers to determining a psychological state or a stress level of the user wearing the wearable device 101 based on the biometric information acquired through the sensor module 201 and providing deep touch pressure to the user by injecting air into the air tube 110 according to the determined psychological state or stress level. The manner in which air is injected into the air tube 110 may vary depending on the determined psychological state or stress level.

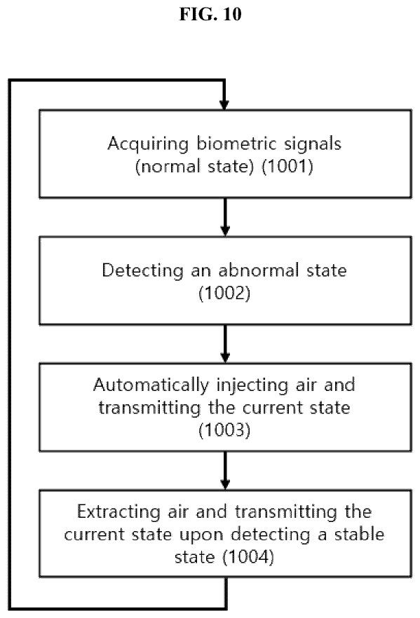

FIG. 10 illustrates a flowchart of a method for caring for a psychological state using biometric information by the wearable device 101 according to one embodiment.

The operations of the wearable device 101 described below may be performed in a different order or simultaneously.

In operation 1001, the wearable device 101 may acquire, in real time, biometric information of a user wearing the wearable device 101 through the sensor module 201. For example, the wearable device 101 may detect an air pressure inside the air tube 110 of the wearable device 101 through the piezoelectric element 402 of the sensor module 201, and may acquire air pressure data. The air pressure may vary according to the user's cardiac activity or respiratory state.

In one embodiment, the wearable device 101 may process the air pressure data into biometric data through the data processing unit 409 and the AI model unit 408. The air pressure data may be understood as raw data or ballistocardiogram (BCG) data. The wearable device 101 may process the raw data through the data processing unit 409. For example, the wearable device 101 may apply a Fourier transform to the raw data as a first operation and apply a wavelet transform as a second operation, thereby generating transformed data. The data obtained by applying the Fourier transform to the raw data may be referred to as first transformed data, and the data obtained by applying the wavelet transform to the first transformed data may be referred to as second transformed data.

In one embodiment, the wearable device 101 may acquire BCG data and respiration data by applying the Fourier transform and the wavelet transform to the raw data. The BCG data and the respiration data may correspond to the second transformed data.

In one embodiment, to acquire the BCG data (or the second transformed data) from the raw data, the wearable device 101 may perform a Fourier transform by separating a first frequency corresponding to a cardiac rhythm. The wearable device 101 may perform a wavelet transform on the first transformed data, obtained through the Fourier transform, using a wavelet function modeled according to the cardiac rhythm.

In one embodiment, to acquire respiration data (or the second transformed data) from the raw data, the wearable device 101 may perform a Fourier transform by separating a second frequency corresponding to a respiratory rhythm. The wearable device 101 may perform a wavelet transform on the second transformed data, obtained through the Fourier transform, using a wavelet function modeled according to the respiratory rhythm.

In one embodiment, the wearable device 101 may acquire biometric data in real time based on data output from the first AI model 408-1 of the AI model unit 408, after inputting the second transformed data—obtained by applying the Fourier and wavelet transforms—to the first AI model 408-1. The biometric data may correspond to data to which the template matching technique of the first AI model has been applied and may be referred to as data to which a raw data filter has been applied. The biometric data may include filtered BCG data or filtered respiration data.

In operation 1002, the wearable device 101 may detect an abnormal state of the user wearing the wearable device 101 based on the biometric data acquired in real time (e.g., BCG data or respiration data) or based on processed biometric data (e.g., heart rate or heart rate variability (HRV) data). For example, the wearable device 101 may detect the user's abnormal state based on whether the numerical value of the biometric data exceeds a designated threshold or range.

In one embodiment, the wearable device 101 may detect the user's abnormal state based on whether the heart rate data exceeds a designated range. The designated range may be between a first value and a second value and may correspond to a normal heart rate range by age. For example, the normal average heart rate for adults over 20 years old is approximately 70-75 beats per minute, and thus the first value may be 70 bpm and the second value may be 75 bpm. However, the invention is not limited to these exemplary values. When the heart rate data exceeds the designated range, the wearable device 101 may determine that the user is experiencing anxiety or stress.

In one embodiment, the wearable device 101 may detect the user's abnormal state based on whether the heart rate variability (HRV) data exceeds a designated range. The designated range may be between a third value and a fourth value and may correspond to a normal HRV range by age. For example, when the psychological state is stable or the stress level is low, HRV may be high, and the difference between the third value and the fourth value may correspond to a first difference value. When the psychological state is unstable or the stress level is high, HRV may be low, and the difference between the third value and the fourth value may correspond to a second difference value. The first difference value may be greater than the second difference value. When the HRV data exceeds the designated range, the wearable device 101 may determine that the user is experiencing anxiety or stress.

In one embodiment, the wearable device 101 may detect the user's abnormal state based on whether the respiration data exceeds a designated range. The designated range may be between a fifth value and a sixth value and may correspond to a normal respiratory rate range. The fifth value may be 12 breaths per minute, and the sixth value may be 20 breaths per minute. When the respiration data exceeds the designated range, the wearable device 101 may determine that the user is experiencing anxiety or stress.

In operation 1003, when the wearable device 101 detects that the user is experiencing anxiety or stress—that is, when an abnormal state is detected—the wearable device 101 may automatically control the actuator to inject air into the air tube 110. The wearable device 101 may transmit at least one of status information indicating the user's abnormal state or information related to the control of the actuator to an external device (e.g., the server 103 or a user terminal).

In one embodiment, the wearable device 101 may determine at least one of: a type of psychological state, a stress level, or a type of symptom, based on the user's biometric information.

In one embodiment, the psychological state may include anger, sadness, joy, comfort, or anxiety. The stress level may be divided into predetermined stages. For example, the stress level may be classified into level 1 through level 5, and the stress may be understood to increase from level 1 to level 5. The type of symptom may include stress-related symptoms, respiration-related symptoms, pain-related symptoms, depression-related symptoms, or heart-disease-related symptoms.

In one embodiment, the wearable device 101 may select an air-injection method corresponding to the determined type of psychological state, stress level, and type of symptom. The air-injection method may include at least one of: an air-injection intensity, an air-injection speed, an air-injection retention time after injection, or an air-compression region. The wearable device 101 may control the actuator of the wearable device 101 to inject air into the air tube 110 based on the selected air-injection method.

For example, when the user's psychological state is determined to be anxiety, the wearable device 101 may inject air into the air tube 110 at a first pressure and a first speed, and may maintain the injected-air state for a first duration. In another example, when the user's psychological state is determined to be anger, the wearable device 101 may inject air into the air tube 110 at a second pressure greater than the first pressure and a second speed faster than the first speed, and may maintain the injected-air state for a second duration longer than the first duration. The air-injection method is not limited to the above examples.

In operation 1004, the wearable device 101 may detect that the user's psychological state is a stable state based on the user's real-time biometric information. For example, when the values of the user's biometric data fall within a designated range, the wearable device 101 may determine that the user's psychological state is stable. For instance, the wearable device 101 may determine that the user's psychological state is stable when at least one of ballistocardiogram (BCG) data, heart-rate data, heart-rate-variability (HRV) data, or respiration data falls within the designated range.

In one embodiment, when the wearable device 101 determines that the user's psychological state is stable, the wearable device 101 may control the actuator to release air from the air tube 110. The wearable device 101 may transmit at least one of status information indicating that the user's psychological state is stable or information related to controlling the actuator to an external device (e.g., the server 103 or a user terminal).

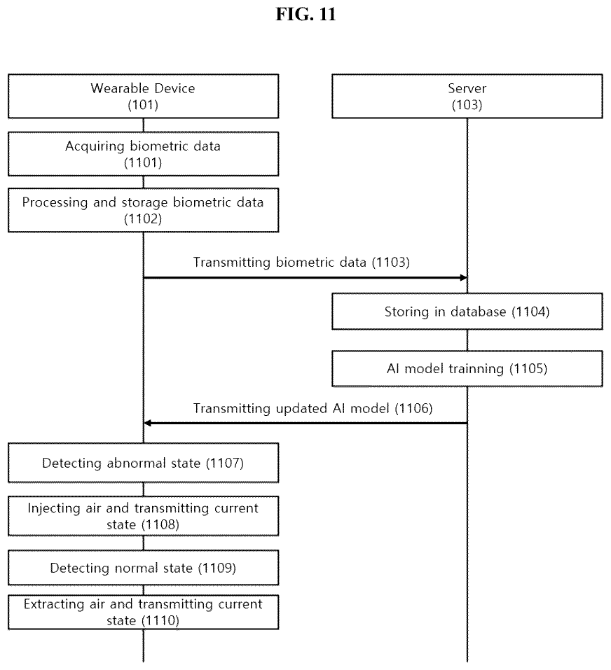

FIG. 11 illustrates a flowchart of operations between the wearable device 101 and the server 103 for caring for the psychological state of a user based on biometric information, according to an embodiment. The operations of the wearable device 101 and the server 103 described below may be performed in a different order or simultaneously.

In operation 1101, the wearable device 101 may acquire biometric data through a sensor provided in the wearable device 101 (e.g., the sensor module 201). The biometric data may include at least one of ballistocardiogram (BCG) data, heart-rate data, heart-rate-variability (HRV) data, respiration data, electrodermal-activity data, photoplethysmogram data, pulse data, or thermal data. The biometric data may refer to processed data obtained by processing raw data (or pressure data or air-pressure data) detected by the piezoelectric element 402 of the sensor module 201.

In one embodiment, the wearable device 101 may acquire air-pressure data (or pressure data or raw data) representing the air pressure inside the air tube 110 of the wearable device 101 through the piezoelectric element 402 of the sensor module 201. The wearable device 101 may process and handle the air-pressure data through the data-processing unit 409 and the artificial-intelligence model unit 408 to obtain biometric data.

In operation 1102, the wearable device 101 may store the biometric data in the data-storage unit 405. The wearable device 101 may perform noise-removal operations on the biometric data through the noise-removal unit 410. In one embodiment, the wearable device 101 may remove noise included in the sensing data (e.g., pressure data, air-pressure data) detected by the piezoelectric element 402 through the noise-removal unit 410.

For example, the wearable device 101 may remove noise in the sensing data by overlapping the sensing data with movement data detected by the motion sensor 407 and eliminating amplified signals generated therefrom. The movement data may include x-, y-, and z-axis data of the accelerometer.

In operation 1103, the wearable device 101 may transmit the biometric data to the server 103 through the communication unit 406. Although not illustrated in FIG. 11, the wearable device 101 may transmit, in addition to the biometric data, at least one of raw data, pre-processed data (e.g., first transformed data or second transformed data), and filtered data processed using the first artificial intelligence model. The raw data may refer to pressure data or air-pressure data. To transmit biometric data to the server 103 through the communication unit 406, the sensor module 201 or the driving unit of the wearable device 101 may be required to be in a charging state. For example, only when the sensor module 201 or the driving unit of the wearable device 101 is in a charging state may the wearable device 101 perform data communication with the server 103 through the communication unit 406.

In one embodiment, the wearable device 101 may further transmit the user's personal information (e.g., age, gender, height, weight, degree of disability, etc.) to the server 103 through the communication unit 406.

In operation 1104, the server 103 may store the biometric data and personal information received from the wearable device 101 in a database. The server 103 may receive the biometric data and personal information from the wearable device 101 only when a wireless communication connection is established between the wearable device 101 and the server 103.

In operation 1105, the server 103 may train an artificial intelligence model using the received biometric data and personal information. The artificial intelligence model may be substantially identical to the artificial intelligence models operating in the wearable device 101 (e.g., the first artificial intelligence model 408-1 and the second artificial intelligence model 408-2).

In one embodiment, the server 103 trains and operates a third artificial intelligence model that performs a raw-data filtering function, extracting filtered data from pre-processed data (e.g., data processed by Fourier transformation and wavelet transformation) using a template-matching technique. The third artificial intelligence model may be trained and operated in a manner substantially identical to the first artificial intelligence model 408-1. The third artificial intelligence model, which applies a template-matching technique, may be referred to as a signal-matching model. For example, the third artificial intelligence model may be trained to output filtered data when raw data are input, using a normal BCG dataset (or a normal biometric dataset) as training data.

In one embodiment, the server 103 extracts features from the raw data received from the wearable device 101, performs vector quantization, and generates a template (or template data) used for applying the template-matching technique.

In one embodiment, the server 103 trains and operates a fourth artificial intelligence model that determines the user's psychological state, stress level, and type of symptom using the user's personal information and biometric information. The fourth artificial intelligence model may be trained and operated in a manner substantially identical to the second artificial intelligence model 408-2. The fourth artificial intelligence model, which is used to classify the user's symptoms, may be referred to as a personalized learning model. For example, the fourth artificial intelligence model may be trained to output a symptom type when receiving, as input, processed data (e.g., heart-rate-variability data, respiration data) derived from the data (e.g., ballistocardiogram data) output by the third artificial intelligence model.

In operation 1106, the server 103 may transmit the generated template (or template data) to the wearable device 101. The server 103 may also update the artificial intelligence models (e.g., the third and fourth artificial intelligence models) trained using the data received from the wearable device 101 (e.g., raw data, biometric data), and transmit the updated models to the wearable device 101.

In operation 1107, which may be substantially identical to operation 1002, the wearable device 101 may detect an abnormal state of the user using the updated artificial intelligence models received from the server 103. The wearable device 101 may input the pre-processed raw data (e.g., the second transformed data) to the updated first artificial intelligence model 408-1 to output filtered data, process the filtered data, and input the processed data to the updated second artificial intelligence model 408-2 to determine whether the user is in an abnormal state.

In operation 1108, which may be substantially identical to operation 1003, the wearable device 101 may automatically inject air into the air tube 110 in response to detecting that the user is in an abnormal state, and may transmit the user's current state to an external device (e.g., the server 103 or a user terminal).

In operation 1109, which may be substantially identical to the stable-state detection of operation 1004, the wearable device 101 may determine that the user is in a stable state when the measured biometric values fall within a designated range.

In operation 1110, which may be substantially identical to the air-release and state-transmission operation of operation 1004, the wearable device 101 may release air from the air tube 110 when determining that the user is in a stable state, and may transmit at least one of information indicating that the user is in a stable state or information indicating that the driving unit has been controlled to release air from the air tube 110 to an external device (e.g., the server 103 or a user terminal).

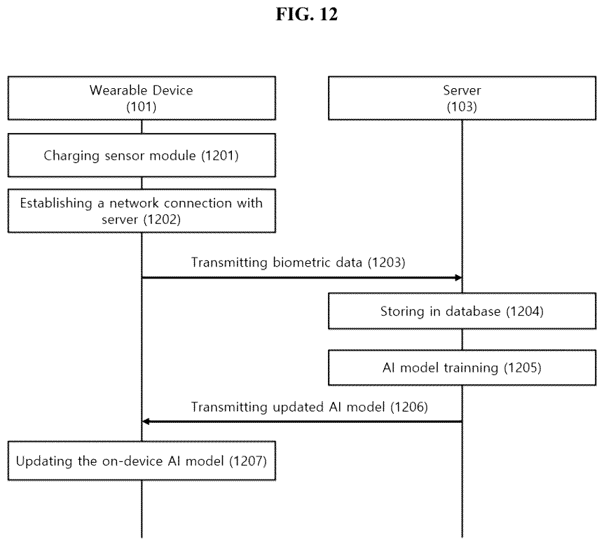

FIG. 12 illustrates a flowchart of operations performed between the wearable device 101 and the server 103 for updating an artificial intelligence model, according to one embodiment. The operations of the wearable device 101 and the server 103 described below may be performed in a different order or simultaneously.

In operation 1201, the wearable device 101 may charge the sensor module 201. The charging of the sensor module 201 may include charging a driving unit that is operably connected to the sensor module 201.

In operation 1202, the wearable device 101 may activate the communication unit 406 only when the wearable device 101 detects that the sensor module 201 (or the driving unit) is in a charging state. That is, until the sensor module 201 (or the driving unit) reaches a charging state, the wearable device 101 may keep the communication unit 406 deactivated. While the communication unit 406 is deactivated, the wearable device 101 may perform data communication among internal circuits of the wearable device 101 using serial communication. The serial communication may include UART, SPI, or I2C communication.

In operation 1203, which may be substantially identical to operation 1103 of FIG. 11, the wearable device 101 may transmit biometric data—obtained based on raw data acquired through the sensor module 201—to the server 103 through the communication unit 406.

In operation 1204, the server 103 may store the biometric data received from the wearable device 101 in a database.

In operation 1205, the server 103 may train an artificial intelligence model based on the biometric data stored in the database, and thereby update the artificial intelligence model. The artificial intelligence model may be substantially identical to the first artificial intelligence model 408-1 and the second artificial intelligence model 408-2 executed on the wearable device 101, and may be an artificial intelligence model trained through active learning.

In operation 1206, the server 103 may transmit the updated artificial intelligence model to the wearable device 101.

In operation 1207, when the wearable device 101 and the server 103 are connected through a network, the wearable device 101 may control its operations using the updated artificial intelligence model received from the server 103.

According to one embodiment, when the network connection between the wearable device 101 and the server 103 is continuously maintained, the wearable device 101 may transmit data collected in real time (e.g., raw data, biometric data, etc.) or may transmit the data at predetermined time intervals to the server 103.

According to another embodiment, when the network connection between the wearable device 101 and the server 103 is continuously maintained, the wearable device 101 may receive the artificial intelligence model updated by the server 103 in real time or at predetermined time intervals.

According to one embodiment, a system for caring for a psychological state of a user based on biometric information of the user may include a wearable device and an external server. The wearable device may include: an air tube; an air inlet provided at one end of the air tube so that air is introduced into or discharged from the air tube; a driving unit that injects or discharges air into or from the air tube through the air inlet; a silicon tube disposed inside a housing of the driving unit and extending from the driving unit to be connected to the air inlet; a sensor module disposed inside the silicon tube and including a piezoelectric element configured to sense pressure; a first communication unit; a first artificial intelligence model unit; and a first processor disposed inside the silicon tube and operatively connected to the driving unit, the sensor module, and the first artificial intelligence model unit. The external server may include a second communication unit, a second artificial intelligence model unit, and a second processor operatively connected to the second communication unit and the second artificial intelligence model unit.

The first processor may acquire air pressure data indicating an air pressure inside the air tube through the piezoelectric element of the sensor module, determine whether a network connection is established so that data communication between the wearable device and the external server is available, and, when it is determined that the network connection between the wearable device and the external server is established, transmit the air pressure data to the external server through the first communication unit. The second processor may receive the air pressure data from the wearable device through the second communication unit, input the air pressure data to a first artificial intelligence model and a second artificial intelligence model included in the second artificial intelligence model unit, update the first artificial intelligence model by training the first artificial intelligence model so as to extract filtered data from the air pressure data, update the second artificial intelligence model by training the second artificial intelligence model so as to determine a state of the user, and, when it is determined that the network connection between the wearable device and the external server is established, transmit the updated first artificial intelligence model and the updated second artificial intelligence model to the wearable device through the second communication unit. The first processor may receive the updated first artificial intelligence model and the updated second artificial intelligence model from the external server through the first communication unit, and determine the state of the user based on values obtained by processing the air pressure data using the updated first artificial intelligence model and the updated second artificial intelligence model.

According to one embodiment, the wearable device may further include a data processing unit configured to process data acquired through the piezoelectric element. The first processor may generate transformed data by sequentially applying a Fourier transform to the air pressure data and a wavelet transform to data obtained by performing the Fourier transform through the data processing unit, and, when it is determined that the network connection between the wearable device and the external server is established, may transmit the transformed data to the external server through the first communication unit.

According to one embodiment, the first processor may apply a first Fourier transform to the air-pressure data through the data processing unit, wherein the first Fourier transform may be a transform that separates a first frequency corresponding to a cardiac rhythm from the air-pressure data. The first processor may further apply a second Fourier transform to the air-pressure data through the data processing unit, wherein the second Fourier transform may be a transform that separates a second frequency corresponding to a respiratory rhythm from the air-pressure data. Based on the first Fourier transform and the second Fourier transform, the first processor may acquire ballistocardiogram (BCG) data and respiration data of the user from the air-pressure data.

According to one embodiment, the first processor may perform a first wavelet transform on the air-pressure data, to which the first Fourier transform has been applied, by using a first wavelet function modeled according to the cardiac rhythm, and may perform a second wavelet transform on the air-pressure data, to which the second Fourier transform has been applied, by using a second wavelet function modeled according to the respiratory rhythm. Based on the first wavelet transform and the second wavelet transform, the first processor may generate transformed data from the air-pressure data, and the transformed data may include the user's BCG data and respiration data.

According to one embodiment, the first artificial intelligence model unit may include a third artificial intelligence model trained to extract filtered data from the transformed data-obtained by performing the first wavelet transform and the second wavelet transform—by using a template-matching technique. When it is determined that the wearable device and the external server are in a state in which the network connection is established, the first processor may update the third artificial intelligence model based on the updated first artificial intelligence model received through the first communication unit.

According to one embodiment, the artificial intelligence model unit may include a fourth artificial intelligence model trained to determine a state of the user based on the transformed data. When it is determined that the wearable device and the external server are in a state in which the network connection is established, the first processor may update the fourth artificial intelligence model based on the updated second artificial intelligence model received through the first communication unit.

According to one embodiment, the first processor may activate the first communication unit when at least one of the sensor module or the driving unit transitions into a charged state, and may determine that the wearable device and the external server have established the network connection.

According to one embodiment, the wearable device may further include a motion sensor. The first processor may acquire movement data of the user through the motion sensor, and may remove noise from the air-pressure data based on the movement data.

What is claimed is:

1. A system for caring for a psychological state of a user based on biometric information of the user, the system comprising:
a wearable device and an external server;
wherein the wearable device comprises:
an air tube;
an air inlet provided at one end of the air tube and configured to allow air to be injected into or discharged from the air tube;
a driving unit configured to inject air into or discharge air from the air tube through the air inlet;
a silicon tube disposed inside a housing of the driving unit and extending from the driving unit to be connected to the air inlet;
a sensor module including a piezoelectric element positioned inside the silicon tube and configured to sense pressure;
a first communication unit;
a first artificial intelligence (AI) model unit; and
a first processor positioned inside the silicon tube and operatively coupled to the driving unit, the sensor module, and the first AI model unit,
and wherein the external server comprises:
a second communication unit;
a second AI model unit; and
a second processor operatively coupled to the second communication unit and the second AI model unit,
wherein the first processor is configured to:
acquire air-pressure data indicating an air pressure inside the air tube through the piezoelectric element of the sensor module;
determine whether a network connection is established between the wearable device and the external server to enable data communication therebetween; and
when the network connection is determined to be established, transmit the air-pressure data to the external server through the first communication unit,
wherein the second processor is configured to:
receive the air-pressure data from the wearable device through the second communication unit;
input the air-pressure data into a first AI model and a second AI model included in the second AI model unit, update the first AI model by training the first AI model to extract filtered data from the air-pressure data, and update the second AI model by training the second AI model to determine a state of the user; and
when the network connection is determined to be established, transmit the updated first AI model and the updated second AI model to the wearable device through the second communication unit,
and wherein the first processor is further configured to:
receive the updated first AI model and the updated second AI model from the external server through the first communication unit; and
determine the state of the user based on values obtained by processing the air-pressure data using the updated first AI model and the updated second AI model.

2. The system of claim 1,
wherein the wearable device further comprises a data processing unit configured to process data acquired through the piezoelectric element, and
wherein the first processor is configured to:
generate transformed data by sequentially applying a Fourier transform to the air-pressure data and a wavelet transform to data obtained through the Fourier transform; and
when the wearable device and the external server are determined to be in an established network connection, transmit the transformed data to the external server through the first communication unit.

3. The system of claim 2,
wherein the first processor is further configured to:
apply a first Fourier transform to the air-pressure data through the data processing unit, the first Fourier transform being a transform that separates a first frequency corresponding to a cardiac rhythm from the air-pressure data;
apply a second Fourier transform to the air-pressure data through the data processing unit, the second Fourier transform being a transform that separates a second frequency corresponding to a respiratory rhythm from the air-pressure data; and
obtain ballistocardiography (BCG) data and respiration data of the user from the air-pressure data based on the first and second Fourier transforms.

4. The system of claim 3,
wherein the first processor is further configured to:
perform a first wavelet transform on the air-pressure data to which the first Fourier transform has been applied, using a first wavelet function modeled according to the cardiac rhythm;
perform a second wavelet transform on the air-pressure data to which the second Fourier transform has been applied, using a second wavelet function modeled according to the respiratory rhythm;
generate the transformed data from the air-pressure data based on the first and second wavelet transforms; and
wherein the transformed data includes the ballistocardiography data and the respiration data of the user.

5. The system of claim 4,
wherein the first AI model unit includes a third artificial intelligence model trained to extract filtered data from the transformed data—obtained through the first and second wavelet transforms—by applying a template matching technique, and
wherein the first processor is configured to update the third artificial intelligence model based on the updated first artificial intelligence model received through the first communication unit when the wearable device and the external server are determined to be in an established network connection.

6. The system of claim 4,
wherein the first AI model unit includes a fourth artificial intelligence model trained to determine a state of the user based on the transformed data, and
wherein the first processor is configured to update the fourth artificial intelligence model based on the updated second artificial intelligence model received through the first communication unit when the wearable device and the external server are determined to be in an established network connection.

7. The system of claim 1,
wherein the first processor is configured to activate the first communication unit and determine that the wearable device and the external server have established the network connection when at least one of the sensor module or the driving unit transitions to a charging state.

8. The system of claim 1,
wherein the wearable device further comprises a motion sensor, and
wherein the first processor is configured to:
acquire movement data of the user through the motion sensor; and
remove noise from the air-pressure data based on the movement data.
